(19) United States
(12) Patent Application Publication
(10) Pub. No.: US 2024/0091434 A1
(43) Pub. Date: Mar. 21, 2024

(54) METHODS, SYSTEMS, AND APPARATUS FOR PREPARING AND/OR ADMINISTERING A GENE THERAPY PRODUCT

(71) Applicant: Regina E. Herzlinger, Belmont, MA (US)

(72) Inventor: Regina E. Herzlinger, Belmont, MA (US)

(21) Appl. No.: 18/368,218

(22) Filed: Sep. 14, 2023

Related U.S. Application Data
(60) Provisional application No. 63/407,557, filed on Sep. 16, 2022.

(51) Int. Cl. A61M 5/142

(52) U.S. Cl. CPC A61M 5/14212

(57) ABSTRACT

Presented herein are methods, systems, and apparatus for performing one or more steps of a gene therapy using an infusion device, e.g., a rapid infusion device, e.g., for the treatment of a disease or condition. For example, it is presented herein that a reduction in the time required to perform one or more of the following steps of a gene therapy—(i) return of red blood cells and plasma to the subject following harvesting of T cells, as part of leukapheresis; (ii) delivery of chemotherapy to the subject to avoid later rejection of the CAR T-cells by the immune system, and/or (iii) administration of genetically modified T-cells to the subject via infusion—can be achieved using the rapid infusion systems, methods, and/or devices described herein.

### CROSS-REFERENCE TO RELATED APPLICATIONS

This application claims the benefit of U.S. Provisional Patent Application No. 63/407,557 filed on Sep. 16, 2022, the text of which is incorporated herein by reference in its entirety.

### FIELD

The subject matter described herein relates to methods, systems, and apparatus for preparing a gene therapy product and/or administering a gene therapy product to a subject, particularly for use in the treatment of a disease or condition.

### BACKGROUND

Gene therapy involves the introduction of genetic material into cells to make a beneficial protein and/or otherwise compensate for abnormal genes, for example, in the treatment of a disease or condition. The genetic material may be introduced into cells of a subject, for example, using a viral vector (e.g., adeno-associated virus (AAV), naked DNA (DNA not associated with proteins, lipids, or other molecules to protect it), or other technique. Following administration of the gene therapy to a subject, the expression of the gene at issue may be either advantageously suppressed or enhanced, and the temporal or spatial pattern of the expression of the gene may be modulated.

One type of cell-based gene therapy is CAR T-cell or CAR-T therapy, which refers to the use of autologous or allogeneic T cells engineered with chimeric antigen receptors (CARs, also known as chimeric immunoreceptors, chimeric T cell receptors, or artificial T cell receptors) as a therapeutic agent in the treatment of a disease, for example, cancer. CAR T-cell therapy generally begins by a healthcare provider collecting blood from a subject to obtain T cells from the subject, and returning remaining blood to the subject. This may involve use of a catheter in a vein in the subject's neck or other location to obtain blood, filtration of the blood to extract white blood cells, and returning of red blood cells and plasma back to the subject (leukapheresis). Then, the T cells are separated and removed from the blood and genetically altered (e.g., ex vivo, in a laboratory) to have chimeric antigen receptors (CAR) inserted, then the genetically altered CAR T-cells are grown in sufficient quantities and introduced into the bloodstream of the subject by intravenous (IV) infusion. The administered CAR T-cells then have a therapeutic effect for the subject, e.g., they bind to and kill cancer cells in the body of the subject. When used for cancer treatments, the subject may be first administered chemotherapy (e.g., also by IV infusion) to avoid rejection of the CAR T-cells by the immune system, followed by administration of the CAR T-cells via infusion.

Another kind of cell-based gene therapy is RNA therapy, which uses pieces of RNA to interact with messenger RNA (mRNA) to affect the amount of protein produced from a gene. Examples of RNA therapy include RNA aptamer therapy, antisense oligonucleotide (ASO), small interfering RNA (siRNA), and microRNA (miRNA) therapies. A further kind of cell-based gene therapy is epigenetic therapy which influences epigenetic changes in cells, such as tags or other modifications that turn a gene on or off, therapy affecting protein production from the gene.

Another type of gene therapy is referred to as CRISPR (clustered regularly interspaced short palindromic repeats) or CRISPR-Cas9, which involves editing genomes to correct genetic defects, allowing for alterations of DNA sequences to modify gene function. CRISPR gene therapy may begin by first, removing blood from a subject to obtain T cells. Then, CRISPR-edited T cells are made ex vivo (in a laboratory). The CRISPR-edited T cells are grown in sufficient quantities, then introduced into the bloodstream of the subject by intravenous (IV) infusion. The administered CRISPR-edited T cells then bind to and kill cancer cells in the body of the subject.

As seen above, certain gene therapies involve harvesting cells from the blood of a subject, genetically altering the harvested cells (e.g., ex vivo, in a laboratory), then reintroducing the genetically-altered cells to the subject. Where cells are harvested from a subject, the therapy usually takes place over multiple sessions. Other gene therapies, such as adeno-associated virus vector-based gene therapies, use a virus vector to deliver a gene into target cells of a subject, and may be administered in a single session, without harvesting cells from the body of the subject.

Gene therapy products that are currently approved by the U.S. Federal Drug Administration include idecabtagene vicluecel (ABECMA), by Celgene Corporation, a Bristol-Myers Squibb Company; HPC Cord Blood (ALLOCORD) by SSM Cardinal Glennon Children's Medical Center; BREYANZI, by Juno Therapeutics, Inc., a Bristol-Myers Squibb Company, ciltacebtagene autoleucel (CARVYKTI), by Janssen Biotech, Inc., HPC Cord Blood (CLEVECORD) by Cleveland Cord Blood Center; HPC Cord Blood (Ducord) by Duke University School of Medicine; Allogeneiic Cultured Keratinocytes and Fibroblasts in Bovine Collagen (GINTUIT), by Organogenesis Incorporated; HPC cord blood (HEMCORD) by New York Blood Center; HPC cord blood by Clinimmune Labs, University of Colorado Cord Blood Bank; HPC Cord Blood by MD Anderson Cord Blood Bank; HPC Cord Blood by LifeSouth Community Blood Centers, Inc.; HPC Cord Blood by Bloodwords; talimogene laherparepvec (IMLYGIC), by BioVex, Inc., a subsidiary of Amgen Inc.; tisagenlecleucel (KYMRIAH), by Novartis Pharmaceuticals Corporation; Azficel-T (LAVIV), by Fibrocell Technologies; LUXTURNA by Spark Therapeutics, Inc.; Autologous Cultured Chondrocytes on a Porcine Collagen Membrane (MACI) by Vericel Corp.; sipuleucel-T (PROVENGE), by Dendreon Corp.; RETHYMIC by Enzyvant Therapeutics GmbH; STRATAGRAFT by Stratetech Corporation; brexucabtagene autoleucel (TECARTUS), by Kite Pharma, Inc.; axicabtagene ciloleucel (YESCARTA), by Kite Pharma, Incorporated; and onasemnogene abeparvovec-xioi (ZOLGENSMA), by Novartis Gene Therapies, Inc.

Where the gene therapy involves harvesting cells from the blood of a subject, there are multiple steps that require delivery of fluids to the subject—these include, for example, (i) return of red blood cells and plasma to the subject following harvesting of T cells, as part of leukapheresis; (ii) delivery of chemotherapy to the subject to avoid later rejection of the CAR T-cells by the immune system, and (iii) administration of genetically modified T-cells to the subject via infusion. These steps are generally performed using standard intravenous lines with gravity fed administration (drip IV).

### SUMMARY

Presented herein are methods, systems, and apparatus for performing one or more steps of a gene therapy using an infusion device, e.g., a rapid infusion device, e.g., for the treatment of a disease or condition. For example, it is presented herein that a reduction in the time required to perform one or more of the following steps of a gene therapy—(i) return of red blood cells and plasma to the subject following harvesting of T cells, as part of leukapheresis; (ii) delivery of chemotherapy to the subject to avoid later rejection of the CAR T-cells by the immune system, and/or (iii) administration of genetically modified T-cells to the subject via infusion—can be achieved using the rapid infusion systems, methods, and/or devices described herein.

In one aspect, the invention is directed to a disposable infusion set for use with an infusion device (e.g., a rapid infusion device) for administering by intravenous infusion one or more volumes of solution to a subject as part of a gene therapy, each volume of solution comprising one of the following: (i) red blood cells and plasma (e.g., being returned to the subject following harvesting of T cells, as part of leukapheresis); (ii) a chemotherapeutic agent (e.g., being administered to the subject to avoid later rejection of the CAR T-cells by the subject's immune system), (iii) genetically modified and/or grown T-cells (e.g., T-cells that have been harvested from the subject then genetically modified and grown ex vivo), (iv) an adeno-associated virus vector-based gene therapy agent, and (v) other gene therapy agent, wherein the disposable infusion set comprises a tubing line or lines and, optionally, a filter (e.g., an inline filter) (e.g., a transfusion filter) (e.g., a filter of pore size suitable for cell products, e.g., a filter of pore size within a range from 50 microns to 300 microns, e.g., from 150 microns to 260 microns) (e.g., wherein the filter comprises a filter membrane that provides very low binding of cell product to the filter membrane), and wherein the tubing line(s) have, collectively, no greater than 200 cc (cubic centimeters) (e.g., no greater than 150 cc, e.g., no greater than 125 cc, e.g., no greater than 100 cc, e.g., no greater than 80 cc) of a total priming volume plus dead space volume. In certain embodiments, large clots and aggregates are filtered out by the filter, yet the filter is porous enough to ensure an effective transfusion flow rate (e.g., blood cells are allowed to pass through the filter, e.g., blood cells are up to 20 micrometers thick). In certain embodiments, the filter provides for retention of bacteria and/or fungi, and/or the filter provides for elimination of air from the solution passing therethrough.

Unlike normal IV lines with gravity fed administration (drip IV), a rapid infusion device does not need a drip chamber to gauge flow rates, since a software-controlled pump is used to administer the fluids. Thus, in certain embodiments, the disposable infusion set does not include a drip chamber. By eliminating the drip chamber, a rapid infusion device provides for administration of product with reduced agitation of the infusate (e.g., the CAR-T cells or other genetically modified/grown cells), as compared to administration by drip IV. The reduced agitation can help avoid problems due to infusate instability and aggregation, potentially improving efficacy.

In certain embodiments, the filter membrane comprises polysulfone (PS), polyethersulfone (PES), and/or cellulose acetate.

In certain embodiments, the disposable infusion set is configured (e.g., and approved) for use with a rapid infusion device (e.g., an infusion device capable of an infusion rate of at least 2 mL/min, e.g., at least 10 mL/min, e.g., at least 20 mL/min, e.g., at least 30 mL/min, e.g., at least 50 mL/min, e.g., at least 75 mL/min, e.g., at least 100 mL/min, e.g., at least 150 mL/min, e.g., at least 200 mL/min, e.g., at least 250 mL/min, e.g., at least 300 mL/min, e.g., at least 400 mL/min, e.g., at least 500 mL/min; e.g., an infusion device capable of infusion rates from about 2 mL/min to about 1500 mL/min).

In certain embodiments, the tubing line or lines fluidly connect (i) an intravenous (IV) bag or other receptacle containing the volume of solution to a pump capable of administering the volume of solution to the subject at a flow rate faster (e.g., substantially faster) than by gravity alone (e.g., faster than a gravity drip device) and/or (ii) the pump to the subject.

In certain embodiments, the filter provides for retention of bacteria and/or fungi, and/or wherein the filter provides for elimination of air from the solution passing therethrough.

In another aspect, the invention is directed to an infusion device (e.g., a rapid infusion device) for administering to a subject, by intravenous infusion, a volume of solution comprising (i) red blood cells and plasma (e.g., being returned to the subject following harvesting of T cells, as part of leukapheresis), (ii) a chemotherapeutic agent (e.g., being administered to the subject to avoid later rejection of the CAR T-cells by the subject's immune system), (iii) genetically modified and/or grown T-cells (e.g., T-cells that have been harvested from the subject then genetically modified and grown ex vivo), (iv) an adeno-associated virus vector-based gene therapy agent, or (v) other gene therapy agent, the rapid infusion device comprising: a pump (e.g., a roller pump or centrifugal pump); and a disposable infusion set comprising a tubing line or lines and, optionally, a filter (e.g., an inline filter), wherein the tubing line or lines fluidly connect (i) an intravenous (IV) bag or other receptacle containing the volume of solution to the pump and/or (ii) the pump to the subject, wherein the tubing line(s) have, collectively, no greater than 200 cc (cubic centimeters) (e.g., no greater than 150 cc, e.g., no greater than 125 cc, e.g., no greater than 100 cc, e.g., no greater than 80 cc) of a total priming volume plus dead space volume, and wherein the pump is configured such that the pump is capable of administering the volume of solution to the subject at a flow rate faster (e.g., substantially faster) than by gravity alone (e.g., faster than a gravity drip device) (e.g., at a flow rate of at least 10 mL/min or at least 15 mL/min or at least 30 mL/min or at least 50 mL/min) and/or the pump is capable of administering the volume of the solution in no more than 3 hours, or no more than 2 hours, or no more than 1 hour, or no more than 45 minutes, or no more than 30 minutes, or no more than 20 minutes.

In another aspect, the invention is directed to a method for administering to a subject one or more volumes of solution as part of a gene therapy using an infusion device (e.g., a rapid infusion device), the method comprising: administering by intravenous infusion a volume of solution comprising (i) red blood cells and plasma (e.g., being returned to the subject following harvesting of T cells, as part of leukapheresis), (ii) a chemotherapeutic agent (e.g., being administered to the subject to avoid later rejection of the CAR T-cells by the subject's immune system), (iii) genetically modified and/or grown T-cells (e.g., T-cells that have been harvested from the subject then genetically modified and grown ex vivo), (iv) an adeno-associated virus vector-based gene therapy agent, or (v) other gene therapy agent using an infusion device (e.g., a rapid infusion device), wherein the infusion device comprises a pump (e.g., a roller pump or centrifugal pump) and a disposable infusion set (e.g., the disposable infusion set of any one of the embodiments described herein) comprising a tubing line or lines and, optionally, a filter (e.g., an inline filter), wherein the tubing line or lines fluidly connect (i) an intravenous (IV) bag or other receptacle containing the volume of solution to the pump and/or (ii) the pump to the subject, for intravenous delivery of the volume of solution to the subject, wherein the tubing line(s) have, collectively, no greater than 200 cc (cubic centimeters) (e.g., no greater than 150 cc, e.g., no greater than 125 cc, e.g., no greater than 100 cc, e.g., no greater than 80 cc) of a total priming volume plus dead space volume, wherein the pump administers the volume of solution to the subject at a flow rate faster (e.g., substantially faster) than by gravity alone (e.g., at a flow rate of at least 10 mL/min or at least 15 mL/min or at least 30 mL/min or at least 50 mL/min) and/or administration of the volume of solution to the subject is completed in no more than 3 hours, or no more than 2 hours, or no more than 1 hour, or no more than 45 minutes, or no more than 30 minutes.

In certain embodiments, the administering step is a member selected from the group consisting of (i) to (iii) as follows: (i) return of red blood cells and plasma to the subject following harvesting of T cells, as part of leukapheresis; (ii) delivery of chemotherapy to the subject to avoid later rejection of the CAR T-cells by the immune system, and (iii) administration of genetically modified T-cells to the subject via infusion.

In certain embodiments, as part of the gene therapy, an apheresis machine is used to receive blood from a subject and separate the blood into its various components, e.g., plasma, platelets, white blood cells, and/or red blood cells. T cells (a type of white blood cell) are harvested and remaining components of the blood are returned to the subject. In certain embodiments, a rapid infusion device and/or disposable infusion set is/are used to receive the blood from the subject and/or to deliver blood back to the subject following harvesting of T cells. A leukocyte adsorber may be used in leukocyte apheresis, e.g., whereby cartridge comprising a hydrophilic membrane (e.g., polysulfone membrane) or other filter separates leukocytes (white blood cells) from the blood. In certain embodiments, various components of the blood filtration systems and methods described in International (PCT) Patent Application No. PCT/US2020/033210, filed May 15, 2020 and published as International Publication No. WO2020/236626; U.S. patent application Ser. No. 17/549,800, filed Dec. 13, 2021; and/or U.S. Provisional Application No. 63/457,898, filed on Apr. 7, 2023, the texts of which are incorporated herein by reference in their entireties, can be used.

### BRIEF DESCRIPTION OF THE DRAWINGS

Drawings are presented herein for illustration purposes, not for limitation. The foregoing and other objects, aspects, features, and advantages of the disclosure will become more apparent and may be better understood by referring to the following description taken in conjunction with the accompanying drawings, in which:

FIG. 1 shows a system and device for administering a solution in one or more steps of a gene therapy via rapid infusion, according to an illustrative embodiment.

FIG. 2 shows another system and device for administering a solution in one or more steps of a gene therapy via rapid infusion, according to an illustrative embodiment.

FIG. 3 shows a method of using devices for administering a solution in one or more steps of a gene therapy via rapid infusion, according to an illustrative embodiment.

FIG. 4 depicts an illustrative disposable infusion set manufactured by Belmont Medical Technologies (3-Spike Disposable Set) (100), originally designed for rapid delivery of warmed blood, with noted design modifications to make the disposable set compatible for use with an infusion device (e.g., a rapid infusion device) for infusion of a volume of solution to a subject in one or more steps of gene therapy, according to illustrative embodiments of the present disclosure.

FIG. 5 depicts the illustrative disposable infusion set of FIG. 4, with notations depicting priming volume and dead space.

FIG. 6 depicts the illustrative disposable infusion set of FIGS. 4 and 5, modified to remove an unnecessary chamber and superfluous tubing, for purposes of infusion of a solution as part of a gene therapy to a patient, according to an illustrative embodiment.

FIG. 7 depicts steps in an illustrative method of CAR T-cell therapy that utilizes the rapid infusion systems, methods, and/or devices in one or more steps of the therapy, according to an illustrative embodiment.

FIG. 8 depicts steps in an illustrative method of CRISPR/Cas9 therapy that utilizes the rapid infusion systems, methods, and/or devices in one or more steps of the therapy, according to an illustrative embodiment.

### DETAILED DESCRIPTION

It is contemplated that systems, architectures, devices, methods, and processes of the claimed invention encompass variations and adaptations developed using information from the embodiments described herein. Adaptation and/or modification of the systems, architectures, devices, methods, and processes described herein may be performed, as contemplated by this description.

Throughout the description, where articles, devices, systems, and architectures are described as having, including, or comprising specific components, or where processes and methods are described as having, including, or comprising specific steps, it is contemplated that, additionally, there are articles, devices, systems, and architectures of the present invention that consist essentially of, or consist of, the recited components, and that there are processes and methods according to the present invention that consist essentially of, or consist of, the recited processing steps.

It should be understood that the order of steps or order for performing certain action is immaterial so long as the invention remains operable. Moreover, two or more steps or actions may be conducted simultaneously.

The mention herein of any publication is not an admission that the publication serves as prior art with respect to any of the claims presented herein. The Background section may include concepts informed by the embodiments recited in the claims and further described elsewhere in the specification. The discussion of concepts in the Background section is not an admission that the subject matter discussed is prior art.

Documents are incorporated herein by reference as noted. Where there is any discrepancy in the meaning of a particular term, the meaning provided in this document is controlling.

Headers are provided for the convenience of the reader—the presence and/or placement of a header is not intended to limit the scope of the subject matter described herein.

As used herein, the term “subject” or “patient” (used interchangeably) refers to an organism, typically a mammal (e.g., a human), though in some embodiments “subject” or “patient” refers to a non-human animal, e.g., a mammal. In some embodiments, e.g., as set forth herein, a subject is suffering from a disease, disorder or condition (e.g., a human patient or an animal patient). In some embodiments, a subject is a laboratory animal.

Commercially available rapid infusion devices are currently designed to rapidly administer a large volume of plasma, blood, or other fluid to subjects in military or civilian emergency situations, for example, a subject suffering from a traumatic injury such as uncontrolled hemorrhage. These systems typically feature a roller pump, centrifugal pump, or other pump mechanism, often with a warmer or other temperature control device. Examples of commercially available rapid infusion systems include the Hotline HL-1200A Rapid Infuser Infusion Pump (capable of infusion rates from 30 mL/min to 1100 mL/min, with maximum rate of 1400 mL/min) (Smiths Group Plc, London, UK); the Belmont® Rapid Infuser RI-2 (capable of infusion rates from 2.5 mL/min to 1000 mL/min), the FMS2000, the buddy™ and the buddy lite™ portable IV & infusion pump (Belmont Medical Technologies, Billerica, MA); LifeFlow Rapid Fluid Infuser, and LifeFlow Plus Rapid Fluid and Blood Infuser (capable of 500 mL of fluid in less than 2 min, 20G IV catheter, or 274 mL/min via 18 ga catheter) (410 Medical, Durham, NC); Thermacor 1200 (capable of infusion rates from 10 mL/hour to 1200 mL/min) (Smisson-Cartledge Biomedical, Macon, GA); The Warrior lite, Warrior, Warrior EXTREME, Warrior Hybrid, and Warrior AC (QinFlow Ltd. of Rosh Ha'ayin Israel); enFlow® IV fluid and blood warming system (CareFusion, Vernon Hills, IL); Medi-Temp by Stryker (Kalamazoo, MI); Ranger by 3M (St. Paul, MN); Level 1 h-1200 Fast Flow Fluid Warmer (Smiths Medical, Dublin, OH); and Thermal Angel® blood and IV fluid infusion warmer (Estill Medical Technologies, Inc., Arlington, TX). Devices with proprietary tubing sets include the enFlow with a 4-mL priming volume and a flow rate up to 200 mL/minute; the Medi-Temp with a flow rate up to 500 mL/minute; and the Ranger by 3M (St. Paul, MN) with a flow rate up to 500 mL/minute. The portable Belmont® buddy™ system is designed for flow rates up to 100 mL/min for crystalloids at 20° C. and up to 50 mL/min for packed red cells at 10° C. The portable, battery powered buddy lite™ system is designed for maximum flow rates of 50-80 mL/min, depending on the input temperature. Pressurized devices for massive transfusion of blood include the Belmont Rapid Infuser RI-2 which can deliver a flow rate of more than 750 mL/minute (e.g., up to 1500 mL/minute); the Level 1 h-1200 Fast Flow Fluid Warmer which can infuse fluids at flows of up to 600 mL/min. Many of the above devices (including the portable devices) include a flow control system and/or other flow and/or metering control devices, such as pressure-regulating valves (PRVs) and/or pressure-responsive valves, to control the specific flow rate of a liquid delivered to the subject and/or to ensure the flow stays below a predetermined maximum flow rate and/or above a predetermined minimum flow rate. Moreover, these flow control devices and/or systems may allow the operator to establish an initial lower flow rate, then increase to a safe higher flow rate if no serious IRRs are observed in the subject.

These rapid infusion systems are not currently used for administration of therapeutics. Rapid infusion systems include those described in any of the following U.S. patents and published patent application, the disclosures of which are incorporated herein by reference: U.S. Pat. Nos. 5,319,170; 6,175,688; 6,236,809; 6,480,257; 7,819,875; 9,737,672; 10,293,099; and 10,485,936; and U.S. Patent Application Publication No. 2009/0192446 (U.S. patent application Ser. No. 12/228,618).

Unlike normal IV lines with gravity fed administration (drip IV), a rapid infusion device does not need a drip chamber to gauge flow rates, since a software-controlled pump is used to administer the fluids. By eliminating the drip chamber, a rapid infusion device provides for administration of gene therapy products with reduced agitation of the cellular products, as compared to administration by drip IV. The reduced agitation can help avoid problems due to instability or aggregation, potentially improving efficacy.

In certain embodiments, the infusion device comprises an elastomeric (e.g., ball) pump, wherein the pump comprises the receptacle containing the volume of solution, and wherein the tubing line or lines fluidly connect (e.g., directly or indirectly) the pump (and, therefore, the receptacle containing the volume of solution) to the subject via the above-described disposable infusion set, for intravenous delivery of the volume of solution to the subject.

The infusion device/system may include an intravenous (IV) bag or other receptacle containing a volume of solution to be administered to the subject. Elements of the infusion device are connected by tubing lines of a disposable set designed for one-time use. The solution is drawn from the IV bag or other receptacle with a pump (e.g., an elastomeric (e.g., ball) pump, a roller pump, or a centrifugal pump). The infusion device may optionally include a heater or other temperature control device. Additionally or alternatively, the infusion device may optionally include one or more of a rate control device (e.g., a pressure-regulating valve, a pressure responsive valve, or the like), one or more sensors, and/or feedback circuitry. The heating element may alternatively or additionally include an air venting mechanism. In certain embodiments, the air venting mechanism is part of the filter (e.g., inline filter).

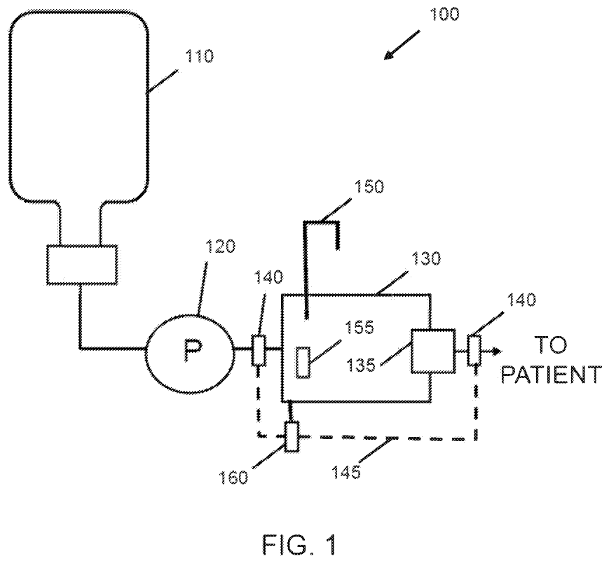

FIG. 1 shows an example of a rapid infusion system 100, in accordance with an illustrative embodiment of the invention. The rapid infusion system 100 includes an intravenous (IV) bag or other receptacle 110 containing a volume of solution to be administered to the patient. Elements of the rapid infusion system 100 are connected by tubing lines (e.g., a disposable set designed for one-time use). The solution is drawn from the IV bag or other receptacle 110 with pump 120 (e.g., a roller pump or centrifugal pump). Element 130 is a heater (which in some embodiments, may be optional) or other temperature control device. Additionally or alternatively, element 130 may optionally include one or more of a rate control device (e.g., a pressure-regulating valve 135, a pressure responsive valve 135, or the like), one or more sensors 140, and/or feedback circuitry 145. Heating element 130 may alternatively or additionally include an air venting mechanism 150.

In certain embodiments, element 130 includes (or is) a filter 155 for filtering out particles (e.g., monoclonal antibody aggregates and/or polyclonal antibody aggregates) from the volume of solution prior to (upstream of) delivery of the filtered solution to the patient. In certain embodiments, the filter 155 has a size small enough (e.g., a mesh tight enough) to catch particles.

In certain embodiments, the filter 155 has a size below 170 microns (e.g., below 150 microns, e.g., below 125 microns, e.g., below 100 microns, e.g., below 75 microns, e.g., below 50 microns, e.g., below 40 microns, e.g., below 30 microns, e.g., below 20 microns, e.g., below 10 microns, e.g., below 8 microns, e.g., below 5 microns, e.g., below 4 microns, e.g., below 2 microns, e.g., below 1 micron, e.g., below 0.7 micron, e.g., below 0.5 micron, e.g., below 0.3 micron, e.g., about 0.2 μm). A standard filter size for blood administration is generally 170-260 microns, which is designed to trap fragments of cells, clots, or particulate matter that may develop as a result of blood product storage. However, a filter that traps smaller particles may be advantageously used for certain embodiments described herein.

The rapid infusion system 100 may include (e.g., as part or all of element 130, or as a separate element) an alarm 160 that identifies air or any other blockage in the line. The rapid infusion system 100 may include (e.g., as part or all of element 130, or as a separate element) an alarm 160 that identifies when a flow rate is above or below a prescribed rate. In certain alternative embodiments, element 130 is positioned between element 110 (IV bag or other receptacle) and the pump 120. In certain embodiments, element 130 (i.e., the heating element) is positioned downstream of pump 120.

Element 130 may have one or more components, any one or more of which may be in a different position with respect to other elements of the system than pictured in FIG. 1 (e.g., one or more elements of 130, e.g., a filter, may be positioned between IV bag 110 and pump 120, ahead of the pump, or may be part of the intravenous (IV) bag or other receptacle 110).

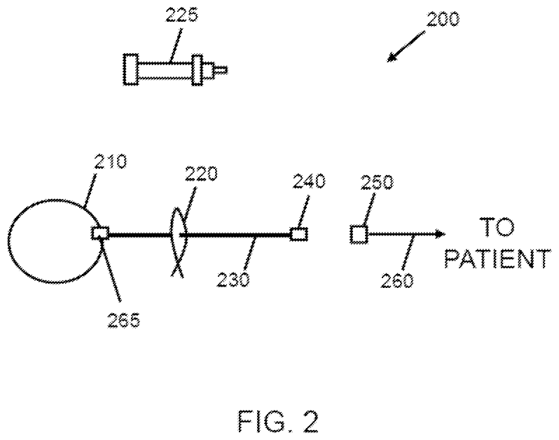

FIG. 2 shows an example of a rapid infusion system 200, in accordance with an illustrative embodiment of the present disclosure. The rapid infusion system 200 shown in FIG. 2 includes an elastomeric medicine ball 210 (also known as a “homeball,” “ball pump,” and/or “grenade pump”). The elastomeric medicine ball 210 may be used for solution delivery in place of the reservoir 110, pump 120, heating element 130, and/or other components illustrated in FIG. 1 and described above. In some embodiments, the system 200 may be used for administering rapid infusion to patients in their own homes, for example. Elastomeric medicine balls 210 are considered pumps, but they do not typically operate with electricity. Elastomeric pumps use pressure created by an elastomeric layer molded into the inside of the medicine ball 210 to infuse its fluid contents into a patient. In certain embodiments, the system 200 includes a pump line 230 that is configured to connect to a patient IV line 260 (that may be already installed (i.e., pre-installed) in the patient, or alternatively may be installed at the time of treatment). Prior to connection with the pump line 230, the patient IV line 260 may be flushed with saline solution (for example, via syringe 225) to ensure no clogs in the system 200, and then subsequently sanitized with alcohol wipes, especially at device access port (or hub) 250 (where contaminants could potentially enter the patient IV line 260). A pump line cap 240 can then be removed and the pump line 230 can be fluidly connected (for example, by inserting and twisting) into hub 250. When the patient is ready for solution delivery, clamp 220 can be removed from the pump line 230, and the drug will begin flowing into the patient via the patient IV line 260.

The elastomeric medicine ball 210, according to certain embodiments of the present disclosure, may be pre-filled with solution (i.e., one or more gene therapy products) and may be pre-pressurized. Once the clamp 220 is removed, the pressure within the elastomeric medicine ball 210 gradually forces the solution out of the elastomeric medicine ball 210, through the pump line 230 and patient IV line 260, and into the patient. In certain embodiments, the delivery process for a single administration can take as long as 90 minutes, but is preferably a shorter time period, for example, administration is completed in no more than 30 minutes (e.g., no more than 25 minutes, e.g., no more than 20 minutes, e.g., no more than 15 minutes, e.g., no more than 10 minutes, e.g., no more than 5 minutes). Elastomer balls generally have a flow restrictor 265 to control the accuracy of the rate of flow. The flow restrictor 265 may be, for example, a steel cannula or a glass capillary molded into system tubing or located inside the elastomeric reservoir. Standard elastomer balls generally provide a flow rate of up to about 250 mL/hr (about 4.17 mL/min). For the methods described herein, elastomer balls may be engineered to permit higher flow rate, for example, flow rate substantially faster than IV flow by gravity alone (e.g., the elastomer ball system provides a flow rate of at least 10 mL/min, or at least 15 mL/min, or at least 20 mL/min, or at least 25 mL/min, or at least 30 mL/min, or at least 35 mL/min, or at least 40 mL/min, or at least 45 mL/min, or at least 50 mL/min). Total solution delivery volumes per elastomeric medicine ball 210 may range up to about 500 mL (e.g., the total volume may be about 50 mL, about 100 mL, about 150 mL, about 250 mL, about 350 mL, about 450 mL, about 500 mL, or within ±50 mL ranges of each of these figures).

In some embodiments, where higher diffusion rates are required, a patient IV line 260 can be installed in each arm (or, alternatively, in one or more other locations of the body), each patient IV line 260 connecting to a separate elastomeric medicine ball 210. In certain embodiments, because the elastomeric medicine ball 210 is calibrated according to the inherent back pressure or resistance in the pump line 230, patient IV line 260, and patient himself/herself, the elastomeric medicine ball 210 generally would not be used in connection with, for example, the fluid heater 130 (shown in FIG. 1). Accordingly, where the contents must be kept refrigerated before use, each elastomeric medicine ball 210 should be removed from the refrigerator with enough time to warm up to room temperature (for example, 10-30 minutes, or about 10-20 minutes) prior to use. However, care should be taken not to expose each elastomeric medicine ball 210 to room temperature for a prolonged period of time, to avoid spoiling and/or breakdown of the drug product.

Still referring to FIG. 2, the system 200 may include one or more elastomeric medicine balls 210 that use only the pressure within each elastomeric medicine ball 210, and not gravity or a separate pump, for solution delivery. As such, patients have the ability to move around and carry the one or more elastomeric medicine balls 210 with them (for example, in a pocket or pockets, etc.) as the drug is flowing. In certain embodiments, once the treatment is complete, each elastomeric medicine ball 210 will be fully deflated, and the pump line 230 can be removed from the device access port 250 (or hub 250). The elastomeric medicine ball 210, pump line 230, clamp 220, and cap 240 can then be disposed of. In certain embodiments, post treatment flushing of the patient IV line 260 should be performed to ensure any drug solution still in the patient IV line at the end of treatment in pushed through the patient IV line 260 into the patient. In certain embodiments, final (i.e., post flushing) sterilization of the hub or device access port 250 should be performed, and the device access port should be capped after sterilization. In some embodiments, heparin may be administered before and/or after the final flushing to avoid clotting, depending on the patient needs. In some embodiments, the system 200 shown in FIG. 2 may also include a heating element in fluid communication with the drug IV line 230 (i.e., downstream of the ball pump 210) to more rapidly heat the infusate. The system 200 may also include an additional pump fluidly upstream of the heating element in order to overcome any addition flow restriction or pressure drop introduced by the heating element.

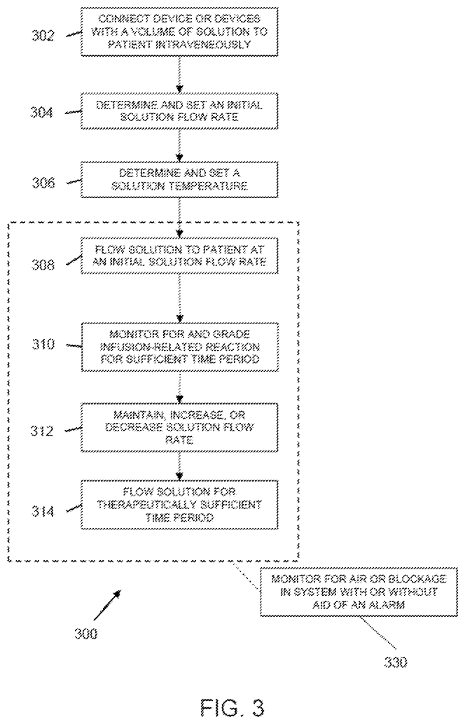

FIG. 3 illustrates a method 300 for systems 100 and/or 200, according to aspects of the present embodiments. Prior to step 302, the method 300 for system 200 may include using a syringe 225 and flushing the intravenously-attached system at port 250 with saline.

Still referring to FIG. 3, in step 304, a solution flow rate may be determined according to various embodiments of system 100 and may be controlled using a flow-controlling device 120. At step 304, an initial solution flow rate may be determined according to various embodiments of system 200 and may be controlled using a flow-controlling device 210. The initial solution flow rate may be 50 mg/hr, 100 mg/hr, or from about 25 mg/hr to about 75 mg/hr, or in other embodiments from about 75 mg/hr to about 125 mg/hr. The flow rate may then be increased in increments of about 25 mg/hr, 50 mg/hr, and/or 100 mg/hr, at time intervals of about 1 minute, 2 minutes, 5 minutes, 10 minutes, 15 minutes, 20, minutes, and/or 30 minutes, to a maximum flow rate of about 400 mg/hr, or from about 300 mg/hr to about 450 mg/hr, or from about 250 mg/hr to about 500 mg/hr, or from about 150 mg/hr to about 450 mg/hr.

Still referring to FIG. 3, in step 306, a solution temperature may be determined according to various embodiments of system 100 and a solution temperature may be set using an optional temperature-controlling device 130. In step 306, a solution temperature may be determined according to various embodiments of system 200 and a solution temperature may be set by equilibrating a solution to an ambient temperature or physiologically-relevant temperature.

Still referring to FIG. 3, in step 308, rapid infusion is started by flowing a solution at an initial flow rate determined by various embodiments of the invention. In step 310, the patient is monitored and graded for infusion-related reactions (IRR).

Still referring to FIG. 3, in step 312, a solution flow rate is maintained, increased, or decreased based on IRR grading and according to various embodiments of the present invention. In step 312, solution flow rate may be maintained for a patient presenting no IRR or presenting a minor IRR after an initial solution flow and being treated using an embodiment of system 100 or an embodiment of system 200. In step 312, solution flow rate may be increased for a patient presenting no IRR or presenting a minor IRR after an initial solution flow and being treated using an embodiment of system 100 or an embodiment of system 200. In step 312, solution flow rate may be increased for a patient presenting no IRR or presenting a minor IRR after an initial solution flow and being treated using an embodiment of system 100 or an embodiment of system 200.

Still referring to FIG. 3, in step 314 a solution may be continued to flow at a flow rate previously determined in method 300 for a time period sufficient for providing the prescribed therapy. At step 330, the method 300 may include monitoring for air and/or blockage in the system (for example, with or without the air of alarm 160) during the entire period of time that solution is flowing (i.e., steps 308-314 in FIG. 3). In some embodiments, prior to step 302, a volume of solution may be loaded into any device or devices (for example, 110, 210, 225) as needed according to aspects of the present disclosure.

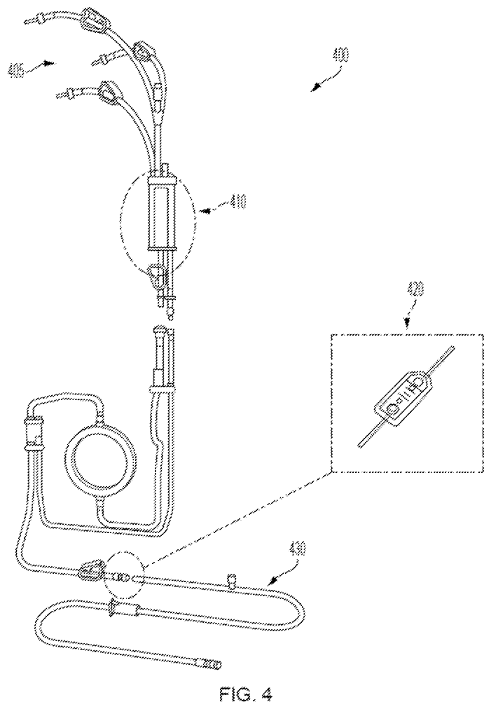

FIG. 4 depicts an illustrative disposable infusion set manufactured by Belmont Medical Technologies (3-Spike Disposable Set) (100), originally designed for rapid delivery of warmed blood, with noted design modifications to make the disposable set compatible for use with an infusion device (e.g., a rapid infusion device) for infusion of a volume of solution to a subject in one or more steps of gene therapy to a subject. The volume of solution may contain (i) red blood cells and plasma (e.g., being returned to the subject following harvesting of T cells, as part of leukapheresis), (ii) a chemotherapeutic agent (e.g., being administered to the subject to avoid later rejection of the CAR T-cells by the subject's immune system), (iii) genetically modified and/or grown T-cells (e.g., T-cells that have been harvested from the subject then genetically modified and grown ex vivo), (iv) an adeno-associated virus vector-based gene therapy agent, or (v) other gene therapy agent, depending on the step of gene therapy being conducted.

In certain embodiments, the existing reservoir chamber and coarse filter (410) is removed in the modified design, and an inline filter (420) is added. In certain embodiments, the filter is a transfusion filter, e.g., a filter of pore size suitable for cell products, e.g., a filter of pore size within a range from 50 microns to 300 microns, e.g., from 150 microns to 260 microns. In certain embodiments, for example, where blood is being returned to the subject via a rapid infusion device, the previous coarse filter (410) is, in fact, used instead of or in addition to the inline filter (420). The inline filter (420) may be positioned, for example, at the connection to a patient line extension (430), as pictured, though other positions may be chosen, and a patient line extension may not be needed. In certain embodiments, the added inline filter (420) also provides air venting. In certain embodiments that use the coarse filter (410), the coarse filter may provide air venting. Furthermore, the circular heat exchanger portion shown in FIG. 4, with high surface area stainless steel rings, may be removed, e.g., where no heating of the delivered solution is required. A pressure chamber and air detector is pictured to the left of the circular heat exchanger portion in FIG. 4, with the fluid path splitting into an infuse line extending from the air detector to the patient line extension (430), and a recirculate line extending below the heat exchanger portion and back up. In certain embodiments, the modified design (e.g., for delivery of a gene therapy product) need not include a recirculate line, and, in certain embodiments, the pressure chamber and/or air detector is/are not needed or is/are positioned elsewhere in the disposable set. The disposable set pictured in FIG. 1 has connections between a heat exchanger, reservoir, and patient line. Where a heat exchanger and/or reservoir is/are not needed, further modifications of this arrangement can be made to adapt the set for use with infusion of treatment solutions.

It is presently found that non-PES coarse blood filters (e.g., 250 μm) such as used with the 120 mL reservoir chamber 410 for infusion of blood or plasma (the original purpose of rapid infusion devices) may clog if used to filter infusions of certain gene therapy products. The inline filter 420 provides retention of undesired particles, bacteria, and fungi, and provides for elimination of air, while avoiding binding of gene therapy products (e.g., CAR-T cells or CRISPR-modified T cells). In certain embodiments, the filter has a membrane made with polysulfone (PS), polyethersulfone (PES), and/or cellulose acetate. Regenerated cellulose has low protein binding but higher than PES and cellulose acetate. Nylon has low to moderate protein binding, and cellulose nitrate has high protein binding.

In certain embodiments, the infusion device (e.g., rapid infusion device) includes a disposable set with a sterile fluid path intended for single-use, with standard luer connectors for connection to a standard catheter and a pressure-regulating valve (PRV) at the input to protect the disposable set and the subject from unintended exposure to high pressure applied to the intravenous (IV) line, wherein the PRV may allow an increase of flow from a low level to a higher level by application of a pressure (e.g., up to 300 mmHg), but will prevent pressure higher than this from reaching the set or IV line distal to it. In certain embodiments, the infusion device also includes a check valve at the output to prevent back flow. In certain embodiments, administration of a therapeutic or other agent is simplified by provision of a portable infusion system (e.g., a portable rapid infusion system) with disposable tubing lines already attached, e.g., where the entire infusion system, pump included, is designed for a single use. Further simplification may be possible by providing the IV bag (or other receptacle) pre-loaded with gene therapy product solution (e.g., pre-made solution) in the appropriate amount and at the appropriate concentration (e.g., all in a self-contained kit). Providing a pre-made solution may not be possible for certain gene therapy solutions.

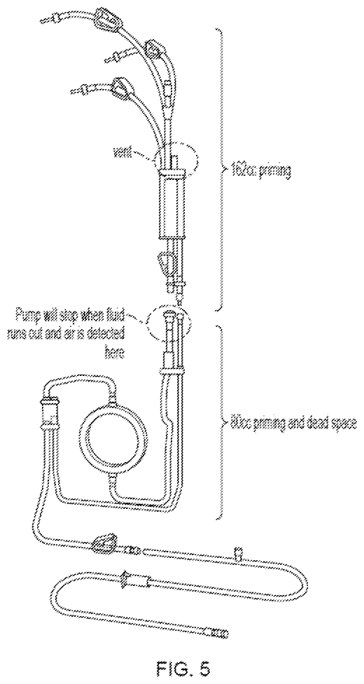

FIG. 5 depicts the illustrative disposable infusion set of FIG. 4, with notations depicting priming volume and dead space. The traditional dead space in the Belmont FMS2000 disposable bag could result in under-dosed infusions if they are infused using the pump without a post infusion saline flush. When medicated solutions are IV infused, the Belmont pump stops infusing once the disposable bag is mostly emptied and air is detected at the inlet of the machine, yet, fluid still remains inside the disposable when the pump stops. The remaining fluid in the “dead space” contains some of the required dose necessary to complete the treatment. The patient will be left under-dosed should the medicated fluid in the “dead space” not be pushed into the patient.

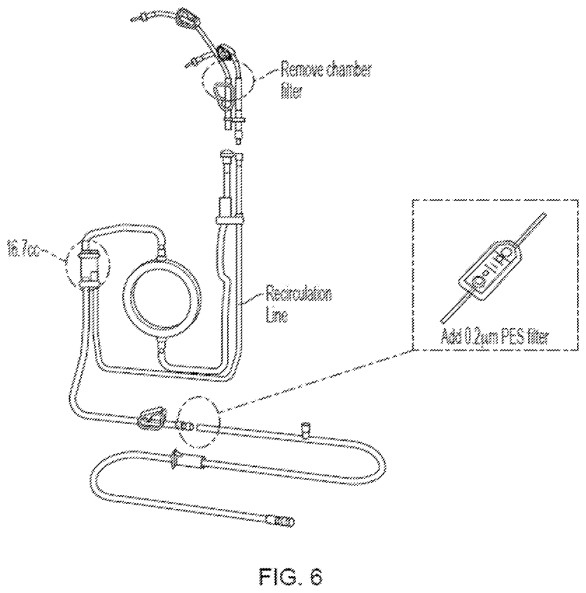

FIG. 6 depicts the illustrative disposable infusion set of FIGS. 4 and 5, modified to remove an unnecessary chamber and superfluous tubing, for purposes of infusion of an antibody treatment to a patient, according to illustrative embodiments of the present disclosure. The modified design eliminates 162 cc of priming fluid that would be needed for blood infusions, but is unnecessary for gene therapy treatment. The unneeded chamber and filter (410) is replaced with minimal tubing and a spike, in this example. The priming volume of the system is 242 cc, which includes a dead volume of only 80 cc as there is a vent at the top of the chamber to empty the camber and associated tubing of 162 cc. FIG. 6 also depicts removal of a chamber filter and addition of a 0.2 μm polyethersulfone (PES) filter, although this modification is optional. FIG. 6 depicts a 16.7 cc volume chamber connecting to the noted recirculation line, for use in an illustrative embodiment. In other embodiments, a different volume is used, or no chamber is used.

In certain embodiments, the pump will stop once the disposable bag is emptied, as detected when air is sensed at the top of the tubing located within the pump housing. When the pump stops, 80 cc of fluid, the so-called “dead space”, remains inside the machine. This volume of fluid contains some of the dose needed to complete the procedure, potentially leaving the patient under-dosed if not flushed.

In certain embodiments, to address the “dead space” issue, after an entire bag of solution is emptied, a saline flush can be performed. For example, a bag of saline (e.g., 100 ml saline bag) can be connected to the unit and infused at the same rate as the solution. This saline flush displaced a substantial portion of the 80 cc of dead space with the saline and deliver the prescribed dose to the patient.

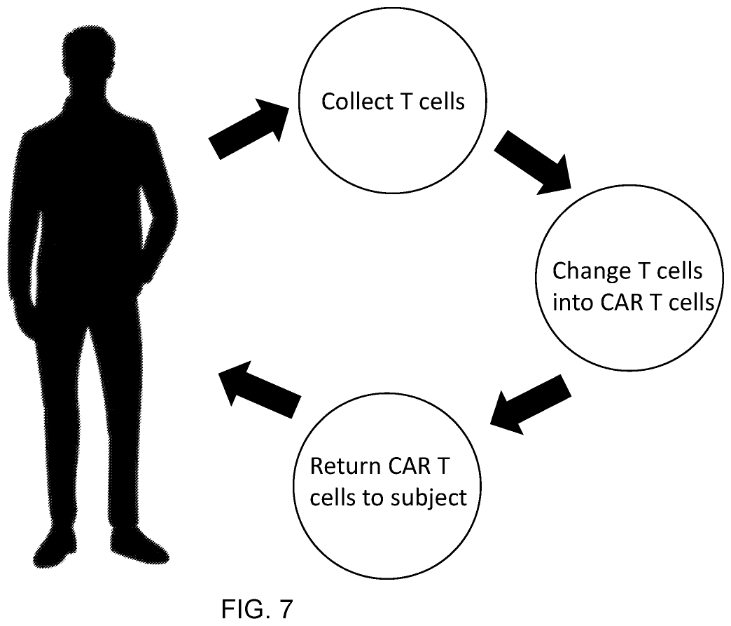

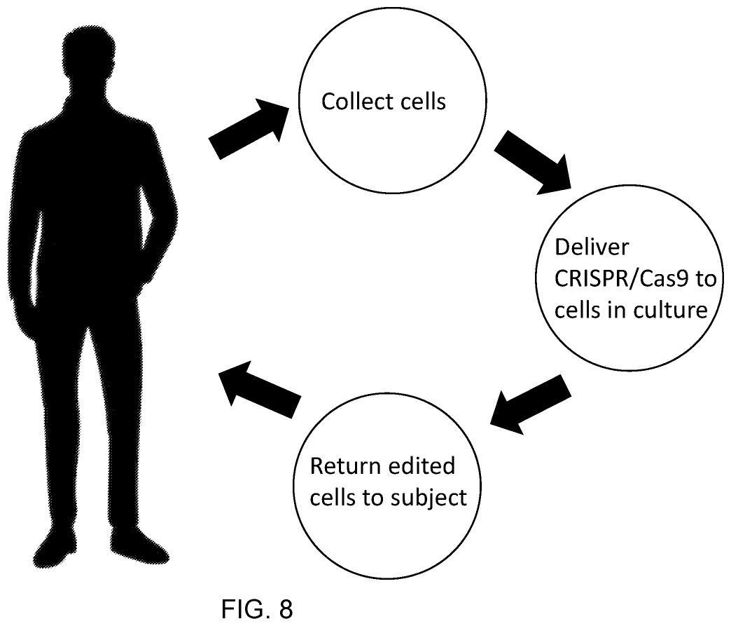

Turning now to FIG. 7 and FIG. 8, these figures depict illustrative methods of administering gene therapy to a patient, for example, CAR T (FIG. 7) and CRISPR/Cas9 therapies, for which one or more steps utilize the rapid infusion systems, methods, and/or devices described herein. Gene therapy involves the introduction of genetic material into cells to make a beneficial protein and/or otherwise compensate for abnormal genes, for example, in the treatment of a disease or condition. The genetic material may be introduced into cells of a subject, for example, using a viral vector (e.g., adeno-associated virus (AAV), naked DNA (DNA not associated with proteins, lipids, or other molecules to protect it), or other technique. Following administration of the gene therapy to a subject, the expression of the gene at issue may be either advantageously suppressed or enhanced, and the temporal or spatial pattern of the expression of the gene may be modulated.

As depicted in FIG. 7, one type of cell-based gene therapy is CAR T-cell or CAR-T therapy, which refers to the use of autologous or allogeneic T cells engineered with chimeric antigen receptors (CARs, also known as chimeric immunoreceptors, chimeric T cell receptors, or artificial T cell receptors) as a therapeutic agent in the treatment of a disease, for example, cancer. CAR T-cell therapy generally begins by a healthcare provider collecting blood from a subject to obtain T cells from the subject, and returning remaining blood to the subject. This may involve use of a catheter in a vein in the subject's neck or other location to obtain blood, filtration of the blood to extract white blood cells, and returning of red blood cells and plasma back to the subject (leukapheresis). Then, the T cells are separated and removed from the blood and genetically altered (e.g., ex vivo, in a laboratory) to have chimeric antigen receptors (CAR) inserted, then the genetically altered CAR T-cells are grown in sufficient quantities and introduced into the bloodstream of the subject by intravenous (IV) infusion. The administered CAR T-cells then have a therapeutic effect for the subject, e.g., they bind to and kill cancer cells in the body of the subject. When used for cancer treatments, the subject may be first administered chemotherapy (e.g., also by IV infusion) to avoid rejection of the CAR T-cells by the immune system, followed by administration of the CAR T-cells via infusion.

Another kind of cell-based gene therapy is RNA therapy, which uses pieces of RNA to interact with messenger RNA (mRNA) to affect the amount of protein produced from a gene. Examples of RNA therapy include RNA aptamer therapy, antisense oligonucleotide (ASO), small interfering RNA (siRNA), and microRNA (miRNA) therapies. A further kind of cell-based gene therapy is epigenetic therapy which influences epigenetic changes in cells, such as tags or other modifications that turn a gene on or off, therapy affecting protein production from the gene.

As depicted in FIG. 8, another type of gene therapy is referred to as CRISPR (clustered regularly interspaced short palindromic repeats) or CRISPR-Cas9, which involves editing genomes to correct genetic defects, allowing for alterations of DNA sequences to modify gene function. CRISPR gene therapy may begin by first, removing blood from a subject to obtain T cells. Then, CRISPR-edited T cells are made ex vivo (in a laboratory). The CRISPR-edited T cells are grown in sufficient quantities, then introduced into the bloodstream of the subject by intravenous (IV) infusion. The administered CRISPR-edited T cells then bind to and kill cancer cells in the body of the subject.

As seen above, gene therapies generally involve harvesting cells from the blood of a subject, genetically altering the harvested cells (e.g., ex vivo, in a laboratory), then reintroducing the genetically-altered cells to the subject. Where cells are harvested from a subject, the therapy usually takes place over multiple sessions. Other gene therapies, such as adeno-associated virus vector-based gene therapies, use a virus vector to deliver a gene into target cells of a subject, and may be administered in a single session, without harvesting cells from the body of the subject.

Where the gene therapy involves harvesting cells from the blood of a subject, there are multiple steps that require delivery of fluids to the subject—these include, for example, (i) return of red blood cells and plasma to the subject following harvesting of T cells, as part of leukapheresis; (ii) delivery of chemotherapy to the subject to avoid later rejection of the CAR T-cells by the immune system, and (iii) administration of genetically modified T-cells to the subject via infusion. Heretofore, these steps have been performed using standard intravenous lines with gravity fed administration (drip IV).

It is presented herein that a reduction in the time required to perform one or more of these steps—(i) return of red blood cells and plasma to the subject following harvesting of T cells, as part of leukapheresis; (ii) delivery of chemotherapy to the subject to avoid later rejection of the CAR T-cells by the immune system, and/or (iii) administration of genetically modified T-cells to the subject via infusion—can be achieved using the rapid infusion systems, methods, and/or devices described herein.

Gene therapy products that are currently approved by the U.S. Federal Drug Administration include idecabtagene vicluecel (ABECMA), by Celgene Corporation, a Bristol-Myers Squibb Company; HPC Cord Blood (ALLOCORD) by SSM Cardinal Glennon Children's Medical Center; BREYANZI, by Juno Therapeutics, Inc., a Bristol-Myers Squibb Company, ciltacebtagene autoleucel (CARVYKTI), by Janssen Biotech, Inc., HPC Cord Blood (CLEVECORD) by Cleveland Cord Blood Center; HPC Cord Blood (Ducord) by Duke University School of Medicine; Allogeneiic Cultured Keratinocytes and Fibroblasts in Bovine Collagen (GINTUIT), by Organogenesis Incorporated; HPC cord blood (HEMCORD) by New York Blood Center; HPC cord blood by Clinimmune Labs, University of Colorado Cord Blood Bank; HPC Cord Blood by MD Anderson Cord Blood Bank; HPC Cord Blood by LifeSouth Community Blood Centers, Inc.; HPC Cord Blood by Bloodwords; talimogene laherparepvec (IMLYGIC), by BioVex, Inc., a subsidiary of Amgen Inc.; tisagenlecleucel (KYMRIAH), by Novartis Pharmaceuticals Corporation; Azficel-T (LAVIV), by Fibrocell Technologies; LUXTURNA by Spark Therapeutics, Inc.; Autologous Cultured Chondrocytes on a Porcine Collagen Membrane (MACI) by Vericel Corp.; sipuleucel-T (PROVENGE), by Dendreon Corp.; RETHYMIC by Enzyvant Therapeutics GmbH; STRATAGRAFT by Stratetech Corporation; brexucabtagene autoleucel (TECARTUS), by Kite Pharma, Inc.; axicabtagene ciloleucel (YESCARTA), by Kite Pharma, Incorporated; and onasemnogene abeparvovec-xioi (ZOLGENSMA), by Novartis Gene Therapies, Inc.

### EQUIVALENTS

While the invention has been particularly shown and described with reference to specific preferred embodiments, it should be understood by those skilled in the art that various changes in form and detail may be made therein without departing from the spirit and scope of the invention as defined by the appended claims.

What is claimed is:

1. A method for administering to a subject one or more volumes of solution as part of a gene therapy using an infusion device, the method comprising:
administering by intravenous infusion a volume of solution comprising a member selected from the group consisting of (i) to (iv) as follows:
(i) red blood cells and plasma being returned to the subject following harvesting of T cells, as part of leukapheresis,
(ii) a chemotherapeutic agent being administered to the subject to avoid later rejection of the CAR T-cells by the subject's immune system,
(iii) genetically modified and/or grown T-cells that have been harvested from the subject then genetically modified and grown ex vivo, and
(iv) an adeno-associated virus vector-based gene therapy agent,
wherein the infusion device comprises a pump and a disposable infusion set comprising a tubing line or lines,
wherein the tubing line or lines fluidly connect (i) an intravenous (IV) bag or other receptacle containing the volume of solution to the pump and/or (ii) the pump to the subject, for intravenous delivery of the volume of solution to the subject,
wherein the tubing line(s) have, collectively, no greater than 200 cc (cubic centimeters) of a total priming volume plus dead space volume, and
wherein the pump administers the volume of solution to the subject at a flow rate of at least 10 mL/min and administration of the volume of solution to the subject is completed in no more than 3 hours.

2. The method of claim 1, wherein the administering step is a member selected from the group consisting of (i) to (iii) as follows: (i) return of red blood cells and plasma to the subject following harvesting of T cells, as part of leukapheresis; (ii) delivery of chemotherapy to the subject to avoid later rejection of the CAR T-cells by the immune system, and (iii) administration of genetically modified T-cells to the subject via infusion.

3. A disposable infusion set for use with an infusion device for administering by intravenous infusion one or more volumes of solution to a subject as part of gene therapy for the subject, each volume of solution comprising a member selected from the group consisting of (i) to (iv) as follows:
(i) red blood cells and plasma being returned to the subject following harvesting of T cells, as part of leukapheresis;
(ii) a chemotherapeutic agent being administered to the subject to avoid later rejection of the CAR T-cells by the subject's immune system;
(iii) genetically modified and/or grown T-cells that have been harvested from the subject then genetically modified and grown ex vivo); and
(iv) an adeno-associated virus vector-based gene therapy agent;
wherein the disposable infusion set comprises a tubing line or lines, and
wherein the tubing line(s) have, collectively, no greater than 200 cc (cubic centimeters) of a total priming volume plus dead space volume.

4. The disposable infusion set of claim 3, wherein the disposable infusion set is configured for use with a rapid infusion device capable of an infusion rate of at least 10 mL/min.

5. The disposable infusion set of claim 3, wherein the tubing line or lines fluidly connect (i) an intravenous (IV) bag or other receptacle containing the volume of solution to a pump capable of administering the volume of solution to the subject at a flow rate faster than by gravity alone and/or (ii) the pump to the subject.

6. The disposable infusion set of claim 3, wherein the set comprises a filter and wherein the filter provides for retention of bacteria and/or fungi, and/or wherein the filter provides for elimination of air from the solution passing therethrough.

7. An infusion device for administering to a subject, by intravenous infusion, a volume of solution comprising a member selected from the group consisting of (i) to (iv) as follows:
(i) red blood cells and plasma being returned to the subject following harvesting of T cells, as part of leukapheresis;
(ii) a chemotherapeutic agent being administered to the subject to avoid later rejection of the CAR T-cells by the subject's immune system;
(iii) genetically modified and/or grown T-cells that have been harvested from the subject then genetically modified and grown ex vivo; and
(iv) an adeno-associated virus vector-based gene therapy agent,
the rapid infusion device comprising:
a pump; and
a disposable infusion set comprising a tubing line or lines,
wherein the tubing line or lines fluidly connect (i) an intravenous (IV) bag or other receptacle containing the volume of solution to the pump and/or (ii) the pump to the subject,
wherein the tubing line(s) have, collectively, no greater than 200 cc (cubic centimeters) of a total priming volume plus dead space volume, and
wherein the pump is configured such that the pump is capable of administering the volume of solution to the subject at a flow rate of at least 10 mL/min and the pump is capable of administering the volume of the solution in no more than 3 hours.

8. The infusion device of claim 7, wherein the device does not comprise a drip chamber.
